(19) United States
(12) Patent Application Publication
(10) Pub. No.: US 2003/0108512 A1
(43) Pub. Date: Jun. 12, 2003

(54) Modified prostaglandin compounds and analogs thereof, compositions containing the same useful for the treatment of cancer

(76) Inventors: Robert Shorr, Edison, NJ (US); Martine Rothblatt, Silver Spring, MD (US); Michael Bentley, Huntsvillef, AL (US); Xuan Zhao, Huntsville, AL (US)

Correspondence Address: Allen R. Kipnes, WATOV & KIPNES, P.C., P.O. BOX 247, PRINCETON JUNCTION, NJ, 08550, US

(21) Appl. No.: 10/006,197

(22) Filed: Dec. 10, 2001

(51) Int. Cl. A61K 31/79; A61K 31/765; A61K 31/557; A61K 31/70

(52) U.S. Cl. 424/78.24; 514/573; 514/23; 424/78.38; 514/59

(57) ABSTRACT

The invention is directed to a pharmaceutical composition containing a cancer-treating effective amount of a prostaglandin compound and analogs thereof having a metabolic rate slowing group attached thereto, and a pharmaceutically acceptable carrier, and methods of employing the same for the treatment of cancer.

### FIELD OF THE INVENTION

The present invention relates to modified prostaglandin compounds, more specifically to long-acting prostaglandin-containing compositions for suitable use in the treatment of cancer.

### BACKGROUND OF THE INVENTION

Prostaglandins are hormone-like substances found in the tissues and organs of the body. No other autocoids or hormones show more numerous or diverse biologically active effects than prostaglandins. They have been found to affect several body systems, including the central nervous, cardiovascular, gastrointestinal, urinary, and endocrine systems. Their effects on the endocrine system include stimulating the release of growth hormone by the pituitary gland, mediating the effects of luteinizing hormone on the ovary, stimulating the dissolution of the corpus luteum, and altering steroid hormone synthesis by the adrenal cortex. One of the prostaglandin compounds has been found to be a powerful stimulant of uterine contractions and may prove useful for inducing labor. However, prostaglandin compounds are short-lived in the body where they are rapidly metabolized by enzymes present in the blood and the lungs and cleared by the kidneys. As a result, most prostaglandin compounds possess a relatively short effective life in the range of from about 3 to 10 minutes.

Prostaglandins, including prostacyclin (PGl₂, a prostaglandin analog), are believed to act on the target cells via cellular surface receptors. These receptors are believed to be part of second messenger systems by which prostaglandin action is mediated. These compounds are known to be responsible in part to regulating a range of physiological responses including, for example, inflammation, blood pressure, blood clotting, fever, pain, induction of labor, and the sleep/wake cycle, and therefore are useful for preventing, controlling and treating a variety of diseases and pathological conditions in warm-blooded animals including humans.

Cancer is a disorder of cell growth that results in invasion and destruction of surrounding health tissue by abnormal cells. Cancer cells typically arise from normal cells whose nature is permanently changed. They often multiply more rapidly than healthy body cells and do not seem subject to normal control by nerves and hormones. They may spread via the bloodstream or lymphatic system to other parts of the body, where they form metastatic clusters or nodules to produce further tissue damage (metastases). The ability of cancer cells to metastasize is a major obstacle in the search for a treatment or cure. The mortality rate of cancer patients is closely linked to recurrence of metastatic cancer cells or malignant tumors. Certain classes of anticancer compounds are capable of inhibiting the spread of malignant tumors by inhibiting one or more steps of the process of tumor migration and dissemination. Such compounds can improve the mortality rate among cancer patients.

The development of a metastasis represents the terminal stage of a complex series of events in which malignant tumor cells, spread to distant sites principally by way of the circulatory system. The first step of metastatic cascade usually involves tumor cell detachment from the primary tumor into newly formed tumor blood vessels. After tumor cell entry into the circulation, tumor cells interact with cellular and non-cellular components of the blood. Thereafter, circulating tumor cells attach to endothelial lining and penetrate into surrounding tissue. Although most tumor cells dispersed through this route die, a small number of tumor cells having inherent biological properties that guarantee their survival, and characterized by high invasiveness, motility, and the ability to avoid detection by the immune system are able to complete all the steps of the metastatic cascade. It is noted that the biochemistry underlying the process of the metastatic cascade is not entirely understood, however, it is believed that surface adhesion proteins, organelles or cell surface structures (e.g. invadopodia) and protease each may play a role in the cascade process. See, for example, Mueller S. C., Ghersi G., Akiyama S. K., Sang Q. X., Howard L., Pineiro-Sanchez M., Nakahara H., Yeh Y., Chen W. T. (1999) J. Biol. Chem. August 27, 274(35): 24947-52; and Chen W. T., Lee C. C., Goldstein L., Bernier S., Liu C. H., Lin C. Y., Yeh Y., Monsky W. L., Kelly T., Dai M. et al (1994) Breast Cancer Res. Treat. 31(2-3): 217-226.

One of these survival-enhancing properties may be the ability to interact with and attach to host platelets in the bloodstream, thus improving their potential to lodge in the microvasculature and adhere to the vascular endothelium lining. Alternatively, once lodged, tumor cells may initiate the formation of surrounding, protective platelet thrombi until extravasation, or infiltration of the tumor cells through the blood vessel walls into surrounding tissue, is completed.

Anticoagulant therapy with aspirin, dipyridamole, heparin, and warfarin has been attempted in the hope of preventing metastasis. However, results to date have been inconclusive. On the other hand, prostaglandin compounds and analogs thereof that are generally known potent anti-thrombogenic agents have been investigated with promising results for possessing potent inhibitory effects on tumor metastasis. Studies have shown that prostaglandin compounds and analogs thereof function primarily by interfering with tumor cell-host interactions (such as tumor cell induced platelet aggregation, tumor cell adhesion to endothelial cells and sub-endothelial matrix, tumor cell induced endothelial cell retraction, etc.) to produce such antimetastatic effects. Such compounds have also been found to exert protective effects in maintaining vascular and platelet homeostasis to deter tumor growth, extravasation, and metastasis.

Further studies performed to date also indicate that prostaglandin and analogs thereof have a spectrum of activity against a wide variety of cancer types. Particularly, many of such prostaglandin and analogs thereof have been shown to possess potent inhibitory effects on tumor cell metastasis in several different animal models including both experimental and spontaneous metastasis models. See, for example, Honn KV et al.: “Prostacyclin: a Potent Antimetastatic Agent”, Science 212: 1270-72 (1981); Carteni et al.: “Biological activity of prostacyclin in patients with malignant bone and soft tissue tumors”, J. Cancer Res. Clin. Oncol. 116 Suppl. Part 1: 631 (1990); Schneider et al.: “Antimetastatic Prostacyclin Analogs”, Drugs Future 18:29-48 (1993); Daneker et al.: “Antimetastatic prostacyclins inhibit E-selectin mediated adhesion of colon carcinoma to endothelial cells”, Journal of Cellular Biochemistry Supplement 19B:25 (1995); and Schirner et al.: “Inhibition of metastasis by Cicaprost in rats with established SMT2A mammary carcinoma growth”, Cancer Detection and Prevention, 21(1): 44-50 (1997). To date, however, the use of prostaglandin and analogs thereof, has been severely limited in the treatment of cancer due largely in part to inherent chemical instability and relatively short effective life as well as limited modes of administration.

Another factor limiting the effective use of prostaglandin and analogs thereof for the treatment of cancer is the difficulty in having such compounds effectively retained by cancerous tumors for a sufficient period of time to achieve a beneficial effect (i.e. there is insufficient passive accumulation of the compound to achieve a desired local concentration). At least part of the reason for such difficulty is that prostaglandin and analogs thereof are relatively small molecules that are able to pass unimpeded through tumor vasculature, and tend to be metabolized and/or excreted rapidly by the host body before any appreciable effect can take place.

Although prostaglandin and analogs thereof hold much promise for use therapeutic agent, there is a need to a) improve the stability of such compounds, b) to extend the effective life of the compounds and c) to enable the compounds to be administered in a more patient friendly dosage regimen than is currently available.

Conjugating biologically active substances such as proteins, enzymes and the like to polymers has been suggested to increase the effective life, water solubility or antigenicity of the active substance in vivo. For example, coupling peptides or polypeptides to polyethylene glycol (PEG) and similar water-soluble polyalkylene oxides (PAO) is disclosed in U.S. Pat. No. 4,179,337, the disclosure of which is incorporated herein by reference. See also, Nucci M., Shorr R. G. L., and Abuchowski A., Advanced Drug Delivery Reviews, 6:133-151, 1991; and Harris J M (ed.), “Polyethylene Glycol Chemistry: Biotechnical and Biomedical Application”, Plenum Press, NY, 1992. Conjugates are generally formed by reacting a therapeutic agent with, for example, a several fold molar excess of a polymer which has been modified to contain a terminal-linking group. The linking group enables the active substance to bind to the polymer. Polypeptides modified in this manner exhibit reduced immunogenicity and antigenicity, and tend to have a higher effective life in the bloodstream than unmodified versions thereof.

To conjugate polyalkylene oxides with an active substance, at least one of the terminal hydroxyl groups is converted into a reactive functional group. This process is frequently referred to as “activation” and the product is called an “activated polyalkylene oxide.” Other substantially non-antigenic polymers are similarly “activated” or “functionalized.”

The activated polymers are reacted with a therapeutic agent having nucleophilic functional groups that serve as attachment sites. Free carboxylic groups, suitably activated carbonyl groups, oxidized carbohydrate moieties and mercapto groups have been used as attachment sites.

It would therefore be a significant advance in the art of drug therapy, especially in the treatment of cancer, if pharmaceutical compositions employing modified prostaglandin and analogs thereof, can be developed with improved chemical stability and an effective life of sufficient duration to enable administration at a reasonable frequency and can be administered in a more patient-friendly manner than current therapies employing unmodified prostaglandin and analogs thereof. It would also be advantageous to provide a pharmaceutical composition that can be administered to a warm-blooded animal including humans, which improves the pharmacokinetic properties of the prostaglandin and analogs thereof in a manner to extend the duration of its inherent anticancer and antimetastatic effects on cancer. It would be a further advance in the art of treating cancer if prostaglandin and analogs thereof could be modified to exhibit increased passive accumulation in tumor cells to provide sufficient local concentrations to effectively treat the cancer without excessive dosing of the compounds.

### SUMMARY OF THE INVENTION

The present invention is generally directed to pharmaceutical compositions comprising modified prostaglandin and analogs thereof that possess pharmaceutical activity suitable for the treatment of cancer.

The present invention is generally directed to novel pharmaceutical compositions comprising modified prostaglandin compounds and analogs thereof including, but not limited to novel modified prostacyclin compounds and analogs thereof which possess activity suitable for the treatment of various types of cancer. The present invention is further directed to methods of using such pharmaceutical compositions for the treatment of cancer. The pharmaceutical compositions of the present invention comprise modified prostaglandin compounds and analogs thereof with improved chemical stability and extended effective life in a warm-blooded animal including humans for effective cancer therapy. Improved stability, effective life and more acceptable modes of administration and dosage regimens are achieved by modifying one or more of the active sites of the prostaglandin compounds and analogs thereof with groups that are capable of stabilizing the compound in vivo.

Thus, in one aspect of the present invention one or more active sites of the prostaglandin compounds or analogs thereof of the present invention are attached to a metabolic slowing group that slows the rate at which the prostaglandin compound is metabolized. A reduction in the metabolic rate provides an increase in the effective life of the active compound, thus a) providing a more efficient administration of pharmaceutical composition comprising the active compound, and b) enabling a more patient-friendly dosage regimen.

In one particular aspect of the present invention, there is provided a pharmaceutical composition useful for the treatment of cancer through inhibition of metastasis and especially by inhibiting attack on the extracellular matrix of normal cells by tumor cells and/or the ability of tumor cells to interact with and attach to host platelets in the blood. The composition comprises a cancer-treating effective amount of a prostaglandin compound and analogs thereof having a metabolic rate slowing group attached thereto and a pharmaceutically acceptable carrier.

In another particular aspect of the present invention, there is provided a method of treating warm-blooded animals including humans afflicted with cancer comprising administering to the warm-blooded animal a therapeutically effective amount of a pharmaceutical composition comprising a cancer-treating effective amount of a prostaglandin compound and analogs thereof having a metabolic rate slowing group attached thereto, and a pharmaceutically acceptable carrier.

In a further aspect of the present invention, there is provided a method of inhibiting metastasis in a warm-blooded animal including humans afflicted with cancer, whereby the method comprises administering to the warm-blooded animal a metastasis inhibiting effective amount of the above-pharmaceutical composition.

In a further aspect of the present invention, there is provided a method of inhibiting collagen degradation induced by metastasizing cancer cells in a warm-blooded animal including humans afflicted with cancer, whereby the method comprises administering to the warm-blooded animal a collagen degradation inhibiting effective amount of the above pharmaceutical composition.

### BRIEF DESCRIPTION OF THE DRAWINGS

The following drawings in which like reference characters indicate like parts are illustrative of embodiments of the invention and are not intended to limit the invention as encompassed by the claims forming part of the application.

FIG. 1 is a graph showing the inhibiting effects on collagen degradation activity of mono-methyl terminated PEG 5000 Da ester-linked Compound V tested over a range of dosages;

FIG. 2 is a graph showing collagen degradation inhibiting activity of Compound V tested over a range of dosages;

FIG. 3 is a graph showing collagen degradation inhibiting activity of a compound of the present invention tested over a range of dosages;

FIG. 4 is a graph showing collagen degradation inhibiting activity of a known hydroxamic acid inhibitor of matrix metalloproteinase, tested over a range of dosages;

FIG. 5 is a graph showing cell toxicity levels of compound V tested over a range of dosages;

FIG. 6 is a graph showing cell toxicity levels of a compound of the present invention tested over a range of dosages;

FIG. 7 is a graph showing cell toxicity levels of a compound of the present invention tested over a range of dosages;

FIG. 8 is a graph showing cell toxicity levels of the known hydroxamic acid tested over a range of dosages;

FIG. 9 is a graph showing apoptosis-inducing activity of compound V tested over a range of dosages;

FIG. 10 is a graph showing apoptosis-inducing activity of a compound of the present invention tested over a range of dosages; and

FIG. 11 is a graph showing apoptosis inducing activity of the known hydroxamic acid tested over a range of dosages.

### DETAILED DESCRIPTION OF THE INVENTION

The present invention is directed to novel pharmaceutical compositions for the treatment of cancer comprising modified prostaglandin compounds and analogs salts and esters thereof in which at least one active site of a prostaglandin compound has attached thereto an inert, non-antigenic, non-immunogenic group having a structure which slows or delays the metabolic rate of the active underlying prostaglandin compound, and which protects the active site when administered to warm blooded animals including humans, to provide a longer effective life and improved sustained release of the active underlying prostaglandin compound.

The inert, non-antigenic, non-immunogenic group provides a transport vehicle for the attached prostaglandin compound from the site of administration to the site of the cancer tissue without material alteration to the cancer-treating beneficial effects of the underlying prostaglandin. As a result more of the cancer-treating compound is available for treating cancer by being present at the target area (cancer sites) for a longer period of time (i.e. the “effective life” of the compound is greater than conventional prostaglandin compounds). As used herein the term “effective life” shall mean the time period during which the present compounds are in their active form in warm-blooded animals including humans. Because more of the active prostaglandin compound is available, dosage regimens can be made less burdensome to the patient.

In the present invention, the inert, non-antigenic, non-immunogenic group conjugates can be formed having hydrolyzable bonds (linkages) between the inert, non-antigenic, non-immunogenic group (e.g. polymer) and a parent or underlying biologically-active moiety, i.e., prostaglandin compound, whereupon administration to the warm-blooded animal including humans, the parent molecule is eventually liberated in vivo. The use of the present compounds enables modifications of the onset and/or duration of action of a biologically-active compound in vivo. The present compounds are typically biologically inert or substantially inactive forms of the underlying or parent compound. The rate of release of the active drug is influenced by several factors including the rate of hydrolysis of the linkage that joins the parent or underlying biologically active compound to the inert, non-antigenic, non-immunogenic group.

Many prostaglandin compounds and analogs thereof, such as prostacyclins and carbaprostacyclins, have been observed to be useful antimetastatic agents against tumor cells. The antimetastatic effects result primarily from the platelet antiaggregatory action and inhibitory effects of prostaglandin compounds and analogs thereof, including prostacyclin, on tumor cell invasion. The above-mentioned antimetastatic effects of prostaglandin compounds and analogs thereof effectively interfere with the ability of tumor cells to interact with and attach to host platelets in the blood and with the ability of the tumor cells to penetrate the extracellular matrix which is a critical factor in tumor metastasis. In this manner, the ability of tumor cells to invade the vascular endothelium is severely compromised and inhibited. Prostaglandin compounds and analogs thereof have also been found to interfere with and disrupt established tumor cells adhering to the endothelium by preventing further formation of surrounding, protective platelet thrombi, thereby preventing eventual extravasation (migration through the endothelium into surrounding tissue mass).

Many of these prostaglandin compounds and analogs thereof, such as prostacyclins and carbaprostacylins, have further been observed to possess apoptotic effects and antiproliferative effects on cancer cells useful for the treatment of cancer.

The protection of at least one active group in accordance with the present invention generally increases the effective life of the prostaglandin compounds and analogs thereof and makes them suitable for various modes of administration as compared to native or unprotected forms of the prostaglandin compounds.

Generally, a significantly lower dosage of the present prostaglandin compounds, in comparison with known native or unconjugated prostaglandin compounds, can be administered to obtain the desired effect, including, but not limited to, inhibiting metastasis, inducing apoptosis in cancer cells, and inducing antiproliferation effects on cancer cells. Because of the rapid metabolism of native or unconjugated forms of known prostaglandin compounds in vivo and the resulting rapid changes in the levels of therapeutic activity which may contribute to stress of the heart, long continuous infusions of relatively large doses of these drugs have been required to maintain an effective blood level in the patient being treated. However, hypotension, tachycardia, and diarrhea, among other side effects, caused by high blood levels of known prostaglandin compounds limit the amount of the known prostaglandin compounds that can be administered. The high cost of prostaglandin compounds also makes it prohibitively expensive to administer such large doses to the patient. The methods of the present invention provide for effective administration of the present prostaglandin compounds, at reduced cost and with reduced side effects.

As used herein the term “prostaglandin compounds and analogs thereof”, hereinafter collectively referred to as “prostaglandin compounds”, shall mean all prostaglandin compounds, and variations thereof which have at least one active group, (e.g., a COOH group and/or an OH group) and which are at least minimally effective for the treatment of cancer in warm-blooded animals including humans. As used herein, the term “present prostaglandin compounds” shall refer to prostaglandin compounds as defined, which have been modified in accordance with the present invention. As used herein, the term “active group” shall mean a site on the prostaglandin compound, which is implicated in the therapeutic effects associated with prostaglandin compounds for the treatment of cancer.

The present invention includes prostaglandin (PG) compounds of all types that are effective for the treatment of cancer because the metabolic rate-slowing group of the present prostaglandin compounds does not materially affect the beneficial activity of the underlying or unmodified prostaglandin compound. For example, the present prostaglandin compounds employed in the present invention include modified PGA, PGB, PGC, PGD, PGE, PGF, and PGI type compounds as well as all subtypes of the foregoing with PGI and PGE and subtypes thereof being more preferred. The prostaglandin compounds can be isolated or extracted from warm-blooded animals or prepared synthetically by techniques known to those of ordinary skill in the art.

All cancers that are capable of metastasizing may be treated in accordance with the present invention including, but not limited to, lung, liver, brain, pancreatic, kidney, prostate, breast, colon, and head-neck cancers.

The preferred pharmaceutical composition comprises a pharmaceutically acceptable carrier and a cancer-treating effective amount of at least one compound possessing exceptional activity for the treatment of cancer, and having the structures of Formulas Ia or Ib

[P-T]ₙ-Z Ia

P-[T-Z]ₙ Ib

wherein

P is a prostaglandin compound or analog thereof, T is an active group of P, and Z is a pharmaceutically acceptable group which is bound to T and which slows the rate at which the prostaglandin compound is metabolized; and

n is an integer of at least 1, and pharmaceutically acceptable salts or esters thereof.

Preferred pharmaceutical compositions comprise a pharmaceutically acceptable carrier and a cancer-treating effective amount of at least one compound having the structure of Formula II

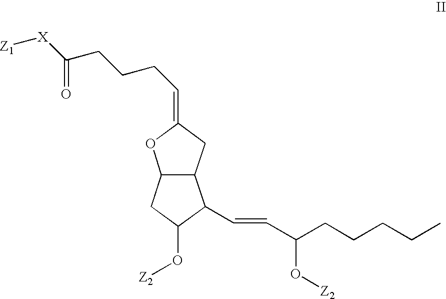

wherein

Z₁ and Z₂ are independently selected from hydrogen and the groups previously defined for Z in Formula I, with the proviso that at least one of Z₁ and Z₂ is not hydrogen; and

X is selected from O or NH; and pharmaceutically acceptable salts or esters thereof.

More highly preferred pharmaceutical compositions comprise a pharmaceutically acceptable carrier and a cancer-treating effective amount of at least one compound represented by Formula II as compounds of Groups 1, 2 and 3 defined below, wherein:

for the Group 1 compounds:

Z₁ is a pharmaceutically acceptable polymer which binds to X and which slows the rate at which the prostaglandin compound is metabolized; and

X is selected from O and NH, and Z₂ is selected from H and an acetyl group;

for the Group 2 compounds:

Z₁ is hydrogen;

X is O, and Z₂ is a pharmaceutically acceptable polymer which slows the rate at which the prostaglandin compound is metabolized and is attached to the oxygen through an ester group; and

for the Group 3 compounds:

Z₁ is a pharmaceutically acceptable polymer as defined in Group 1;

X is O or NH, and Z₂ is a pharmaceutically acceptable polymer as defined in Group 2, attached to the oxygen through an ester group.

Preferred pharmaceutical compositions comprise a pharmaceutically acceptable carrier and a cancer-treating effective amount of at least one compound having the structure of Formula III

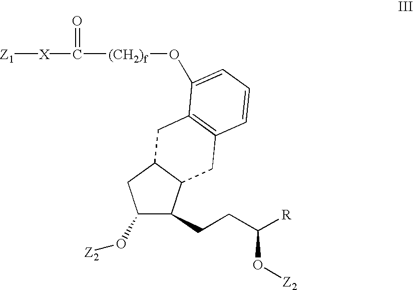

wherein

Z₁ and Z₂ include the same groups as previously defined in Formula II also with the proviso that at least one of Z₁ and Z₂ are not hydrogen;

f is an integer of from 1 to 3;

X is selected from O and NH; and

R is selected from hydrogen and an alkyl group preferably having 1-6 carbon atoms, and pharmaceutically acceptable salts or esters thereof.

More highly preferred pharmaceutical compositions comprise a pharmaceutically acceptable carrier and a cancer-treating effective amount of at least one compound of Groups 4-6 encompassed by Formula III as described below:

for the Group 4 compounds:

Z₁ is a pharmaceutically acceptable polymer that binds to X and which slows the rate at which the prostaglandin compound is metabolized;

X is selected from O and NH, and Z₂ is selected from hydrogen and an acetyl group;

for the Group 5 compounds:

Z₁ is hydrogen, X is O, and Z₂ is an acetyl group, or a pharmaceutically acceptable polymer which slows the rate at which the prostaglandin compound is metabolized and is attached to the oxygen through an ester or ether group;

for the Group 6 compounds:

Z₁ is a pharmaceutically acceptable polymer as defined in Group 4, X is selected from O and NH, and Z₂ is a pharmaceutically acceptable polymer as defined in Group 5.

Highly preferred present prostaglandin compounds are those where the Z₁ and/or Z₂ groups are polyethylene glycols having the formula CH₃OCH₂CH₂(OCH₂CH₂)ₐ, wherein a is from about 1 to 1000.

Particularly preferred pharmaceutical compositions comprise a pharmaceutically acceptable carrier and a cancer-treating effective amount of at least one compound having the structure of Formula IV:

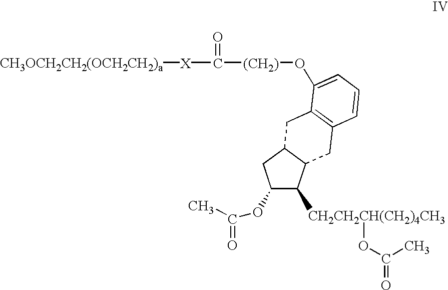

wherein

a and X are as defined above. Preferably, a may range from about 6 to 600, most preferably from about 6 to 460.

The underlying prostaglandin compounds encompassed by the present invention each include a single active COOH group and one or more active OH groups. When at least one of these active groups are protected in the manner described herein, it is possible to more effectively avoid or at least delay enzymatic deactivation and/or excretion before the underlying prostaglandin compound can effectively reach the target area.

In a preferred form of the invention, one or more of the active groups (COOH and OH) of the underlying prostaglandin compounds are linked to linear, branched and/or circular polymers and copolymers that are inert, non-antigenic and non-immunogenic. In addition, the polymers must be capable of separating from the corresponding attached prostaglandin compounds at a rate that is suitable for delivery to the target area of warm-blooded animals including humans in a manner to provide sustained release in the body. To the extent that any of the polymer remains attached to the prostaglandin compound, it should not adversely affect the ability of the underlying prostaglandin compound to treat cancer.

To conjugate prostaglandin compounds to polymers such as polyalkylene oxides, one or more of the hydroxyl groups of the polymer is converted into a reactive functional group enabling attachment of the polymer to the prostaglandin compound.

The activated polymers are reacted with the prostaglandin compound so that attachment of the polymer preferably occurs at the free carboxylic acid groups and/or hydroxyl groups of the prostaglandin compound. Attachment of carbonyl groups, oxidized carbohydrate moieties and mercapto groups, if available, or made available on the prostaglandin compound, are formed as conjugation sites for formation of the present prostaglandin compounds.

In a preferred aspect of the invention, amide or ester linkages are formed between the carboxylic or hydroxyl groups and the activated polyalkylene oxides. Polymers activated with urethane-forming linkers or the like, and other functional groups useful for facilitating the attachment of the polymer to the prostaglandin compound via carboxylic or other groups are encompassed by the present invention.

Among the substantially non-antigenic polymers, polyalkylene oxides (PAO's) especially mono-activated, alkyl-terminated polyalkylene oxides such as polyethylene glycols (PEG) and especially monomethyl-terminated polyethylene glycols (mPEG). It is noted that, in general, each PEG or mPEG is followed by a number which corresponds to its average molecular weight. Bis-activated polyethylene oxides are also contemplated for purposes of cross-linking the prostaglandin compound or providing a means for attaching other moieties such as targeting agents for localizing the polymer-prostaglandin conjugate in the target area such as, for example, the lungs or blood vessels in the extremities.

Suitable polymers especially PEG or mPEG, will vary substantially by weight. Polymers having molecular weights ranging from about 200 to about 80,000 daltons are typically employed in the present invention. Molecular weights from about 2,000 to 42,000 daltons are preferred, and molecular weights of from about 5,000 to 28,000 daltons are particularly preferred.

The polymers preferably employed in the present invention as protective groups are water-soluble at room temperature. A non-limiting list of such polymers includes polyalkylene oxide homopolymers such as PEG and mPEG or polypropylene glycols, polyoxyethylenated polyols, copolymers thereof and block copolymers thereof. In addition to mPEG, C₁₋₄alkyl-terminated polymers are also useful.

As an alternative to PAO-based polymers, effectively non-antigenic materials such as dextran, polyvinyl pyrrolidones, polyaorylamides, polyvinyl alcohols, carbohydrate-based polymers and the like can be used. Modifications of the prostaglandin compounds may further include, but are not limited to, acetylation, carboxylation, glycosylation, phosphorylation, lipidation, and acylation. Those of ordinary skill in the art will realize that the foregoing list is merely illustrative and that all polymer materials having the qualities herein are contemplated.

The prostaglandin compounds are coupled to the protective groups as described to provide present prostaglandin compounds which effectively deliver the active underlying prostaglandin compound to the target area and maintain the prostaglandin compound within the target area for a longer period of time than achieved with known prostaglandin compounds. The present prostaglandin compounds are therefore particularly suited for the treatment of cancer.

Applicants have discovered that there is a relationship between the number of active groups that are protected (i.e. coupled to a protective group as defined herein) and the molecular weight of the protective group as discussed in detail hereinafter.

As previously indicated, many of the known prostaglandin compounds which are used in cancer therapy have a very short effective life in a warm blooded animal, typically less than one hour. In accordance with the present invention, the effective life is increased up to and over several hours. A longer effective life reduces the number of potentially damaging and/or dramatic changes in the levels of the therapeutic agent as well as reduces the number of times that the prostaglandin compounds may be administered. The present prostaglandin compounds may therefore be delivered in lower dosage amounts and with less frequency and less risk to the patient.

The active groups of the prostaglandin compounds include a COOH group and one or more OH groups. One or more of these active groups can be protected by a protective group as will be more specifically set forth hereinafter. The protective groups may generally have a molecular weight of up to 500,000 or more. In a preferred form of the invention, a group of at least 5,000 daltons should be conjugated to the COOH when the OH groups are not protected, more preferably at least 20,000 daltons. It has been observed that protective groups of molecular weight of at least 5,000 daltons can slow excretion of the compounds, thereby contributing to increased effective life in warm-blooded animals including humans.

The protective groups are any groups that serve to protect the active groups (COOH and OH) from premature metabolism but can readily separate from the active groups in a controlled manner for sustained release and/or may remain attached to the active group without adversely affecting the function of the prostaglandin compound. Such protective groups include, for example, polymers, straight and branched chain alkyl groups, aralkyl groups, aryl groups, acyl groups, heterocyclic groups, alkylene groups all of which may be substituted with substituents selected from, for example, alkyl, aryl, aralkyl and the like.

Among the polymers that may be conjugated to the active group include polyglycols, polyvinyl polymers, polyesters, polyamides, polysaccharides, and polymeric acids and combinations thereof. The preferred polyglycols include polyethylene glycol and polypropylene glycol. The preferred polysaccharide is polysaccharide B. Among the polyacids that may be used in accordance with the present invention, are polyamino acids and polylactic acids. The preferred polymers among the classes of polymers mentioned above are polyethylene glycols (PEG). In addition to the polymers mentioned above, such polymers as dextran, cellulosic polymer and starches may be also used in accordance with the present invention.

The polymers may be linked to the active COOH or OH group through a group such as for example, through an amide group, an ester group or the like.

The present invention further provides a method of treating warm-blooded animals including humans afflicted with cancer comprising administering to warm-blooded animals a cancer-treating effective amount of a pharmaceutical composition comprising a pharmaceutically acceptable carrier and a present prostaglandin compound preferably having a structure of Formulas Ia or Ib. As used herein a “cancer-treating effective amount” is defined to be the amount sufficient to bring about therapeutic effects on the cancer to be treated in the warm-blooded animal. The precise amount that is considered effective for a particular therapeutic purpose will, of course, depend upon the specific circumstance of the warm-blooded animal being treated and the magnitude of the effect desired. Titration to effect may be used to determine proper dosage.

The present invention further provides a method of inhibiting metastasis in a warm-blooded animal including humans afflicted with cancer comprising administering to the warm-blooded animal a metastasis inhibiting effective amount of the pharmaceutical composition of the present invention. As used herein a “metastasis inhibiting effective amount” is defined as the amount sufficient to bring about the slowing, halting or preventing of the onset of metastasis by the cancer being treated in the warm-blooded animal. The precise amount that is considered effective for a particular therapeutic purpose will, of course, depend upon the specific circumstances of the warm-blooded animal being treated and the magnitude of the effect desired.

The present invention further provides a method of inhibiting collagen degradation induced by metastasizing cancer cells in a warm-blooded animal including humans afflicted with cancer comprising administering to the warm-blooded animal a collagen degradation inhibiting effective amount of the pharmaceutical composition of the present invention. As used herein a “collagen degradation inhibiting effective amount” is defined as the amount sufficient to bring about the slowing, halting or preventing of the onset of collagen degradation caused by the cancer being treated in the warm-blooded animal. The precise amount that is considered effective for a particular therapeutic purpose will, of course, depend upon the specific circumstances of the warm-blooded animal being treated and the magnitude of the effect desired.

The present prostaglandin compounds are employed as part of a pharmaceutical composition including a pharmaceutically acceptable carrier for the treatment of cancer including, but not limited to, inhibiting metastasis of the cancer, inhibiting collagen degradation induced by metastasizing cancer cells, inducing apoptosis in cancer cells, and inducing antiproliferation effects on cell growth. The compounds employed for this purpose are typically administered in an amount of from 0.1 to 500 mg/kg/day, preferably from about 0.5 to 100 mg/kg/day, and more preferably from about 25 to 35 mg/kg/day. The dosage amount may vary depending upon a number of factors including, but not limited to, the type of cancer treated, the mode of administration, the patient profile (age, weight, etc.) and the like.

The pharmaceutical composition comprising at least one present prostaglandin compound of the present invention may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those known in the art of pharmaceutical formulation.

The present prostaglandin compounds may be administered by any suitable means, for example, orally, such as in the form of tablets, capsules, granules or powders; sublingually; bucally; parenterally, such as subcutaneous, intravenous, intramuscular, or intrasternal injection or infusion techniques (e.g., as sterile injectable aqueous or non-aqueous solution or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present prostaglandin compounds may be based for immediate release or extended release by the use of suitable pharmaceutical compositions comprising the present compounds, or, particularly in the case of extended release, by the use of devices such as subcutaneous implants or osmotic pumps. The present prostaglandin compounds may also be administered in the form of liposomes.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms that may be used.

Exemplary compositions include those formulating the present compound(s) with fast dissolving diluents such as mannitol, lactose, sucrose, and/or cyclodextrins. Also included in such formulations may be high molecular weight excipients such as celluloses (avicel). Such formulations may also include an excipient to aid mucosal adhesion such as hydroxypropyl cellulose (HPC), hydroxypropyl methylcellulose (HPMC), sodium carboxymethyl cellulose (SCMC), maleic anhydride copolymer (e.g. Gantrez), and agents to control release such as polyacrylic copolymer (e.g. Carbopol 934). Lubricants, glidants, flavors, coloring agents and stabilizers may also be added for ease of fabrication and use.

Exemplary compositions for nasal aerosol or inhalation administration include solutions in saline that may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories that may contain, for example, a suitable non-irritating excipient, such as cocoa butter or synthetic glyceride esters, which are solid at ordinary temperatures, but liquify and/or dissolve in the rectal cavity to release the drug.

Exemplary compositions for topical administration include a topical carrier such as Plastibase (mineral oil gelled with polyethylene).

The cancer-treating effective amount of the present prostaglandin compounds may be determined by one of ordinary skill in the art, and includes exemplary dosage amounts for an adult human from about 0.1 to 500 mg/kg, and preferably from about 0.5 to 100 mg/kg of body weight of the present prostaglandin compounds per day, which may be administered in a single dose or in the form of individually divided doses, such as from 1 to 4 times per day. All cancers that are capable of metastasizing may be treated in accordance with the present invention including, but not limited to, cancers of the lung, liver, brain, pancreatic, kidney, prostate, breast, colon, and head-neck. It will be understood that the specific dose level and frequency of dosage for any particular subject may be varied and will depend upon a variety of factors including the activity of the specific compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to heart failure.

The present prostaglandin compounds may be administered subcutaneously in the form of a liquid reconstituted from a lyophilized powder that may additionally contain preservatives, buffers, dispersants, etc. Preferably, the prostaglandin compounds are reconstituted with a medium normally utilized for intravenous injection, e.g., preservative-free sterile water. Administration may be accomplished by continuous intravenous or subcutaneous infusion or by intravenous injection. For continuous infusion, the daily dose can be added to normal saline or other solution and the solution infused by mechanical pump or by gravity.

An assay developed for screening antimetastatic agents and determining the therapeutic response produced by such agents will now be described herein. The corresponding assay has been designed to measure the changes in the invasive and MOM adhesive capacity of the tumor cells brought about by the antimetastatic agent. It is known that tumor cells are capable of adhering to the basement membrane underlying blood vessel walls and entering through the corresponding adjacent connective tissues and extracellular matrix. See Liotta et al., Cancer Metastasis and Angiogenesis: An Imbalance of Positive and Negative Regulation, Cell 64, 327-336 (1991). Circulating tumor cells can adhere to the endothelium at metastatic sites and subsequently invade the extra-cellular matrix composed primarily of collagen, laminin, and fibronectin.

The assay for screening antimetastatic agents is comprised of a collagenous matrix comprising collagenous components including but not limited to Type I or Type VI collagen or denatured collagen such as gelatin to form a scaffold-matrix. The scaffold matrix is subsequently coated with blood-borne components such as fibronectin, laminin, and fibrin for inducing adherence of the tumor cells and thereafter, labeled with quench fluorescent dyes. During performance of the assay, tumor cells that are present, adhere, ingest and invade the labeled scaffold-matrix resulting in the release of highly fluorescent peptides. Generally, tumor cells include a specific cell structure typically comprised of tentacles or arms (i.e. invadopodia) which secrete specific digestive enzymes including seprase, dipeptidyl peptidase IV (DPPIV), membrane Type-1 matrix metalloproteinases, and the like, to breakdown the collagen matrix. The fluorescence becomes visible only in the presence of these specific digestive enzymes produced by the tumor cells and increases in proportion to the activity of the digestive enzymes, thus providing a qualitative/quantitative measure of the invasiveness of the tumor cells. Other labeling methods including, but not limited to the use of, biotin, color dyes, and radioactive probes may also be used.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

### EXAMPLE 1

### Synthesis of mPEG-5 kDa-amide-Compound V

A compound of Group 4 wherein Z₁ is an mPEG with a molecular weight of about 5,000 daltons (Da) referring hereinafter as “mPEG-5 k”, where the number following the mPEG corresponds to its average molecular weight, X is NH and Z₂ is hydrogen was prepared in the following manner.

200 mg of a compound having the Formula V shown below, was obtained from United Therapeutics Corporation of Silver Spring, Md.

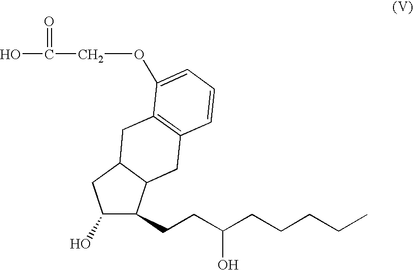

Compound of Formula V was placed into a round bottom flask along with mPEG5 k amine (2.5 g), 2-hydoxybenzyltriazole (HOBT, 67 mg), 4-(dimethylamino)pyridine (DMAP, 61 mg) and dicyclohexylcarbodiimide (DCC, 140 mg). The materials were mixed with 60 ml of anhydrous methylene chloride. The mixture was stirred at room temperature overnight and thereafter the solvent was removed by vaporization. The residue was dissolved in 25 ml of 1,4 dioxane and the insoluble solid was removed by filtration. The solvent was condensed and then precipitated into 100 ml of 50:50/ether:isopropanol. The precipitate was collected by filtration and dried under vacuum. The resulting yield was 2.5 g (93%). ¹H NMR(DMSO-d₆): δ3.5 (br m, PEG), 7.897 (t, —PEGNH—CO-(Compound V)), 4.49 (d, (Compound V)-OH¹), 4.24 (d, (Compound V)-OH²), 0.864 (t, (Compound V)-CH₃), 4.436 (s, (Compound V)-CH₂CONHPEG), 7.045 (t, Compound V aromatic proton), 6.7 (d+d, Compound V aromatic proton).

### EXAMPLE 2

### Synthesis of mPEG5 kDa-ester-Compound V Diacetate

A compound of Group 4 wherein Z₁ is an mPEG with a molecular weight of about 5,000 daltons, or mPEG-5 k, X is O and each Z₂ is an acetyl group, was prepared in the following manner.

In a round-bottom flask, Compound V (400 mg) and pyridine (200 μl) were mixed in 35 ml of anhydrous methylene chloride. 500 μl of acetic anhydride was added to the suspension. The mixture became homogenous in a few hours and the solution was stirred at room temperature overnight. The solvent was condensed and phosphate buffer (0.1 M, pH 7.4) was added to the residue. The mixture was rapidly stirred for 30 minutes, and the mixture was extracted with methylene chloride three times. The combined organic phase was dried over sodium sulfate, and the solvent was removed by vaporization. An oily product, Compound V diacetate, was obtained. The yield was 340 mg (80%). ¹H NMR(DMSO-d₆): 1.91 (s, (Compound V)-O¹COCH₃), 2.00 (s, (Compound V)-O²COCH₃), 0.84 (t, (Compound V)-CH₃).

In a round-bottom flask, mPEG-5 k (3.8 g), Compound V diacetate from the previous step (320 mg), 2-hydroxybenzyltriazole (HOBT, 103 mg), 4-(dimethylamino)-pyridine (DMAP, 93 mg) and dicyclohexylcarbodiimide (DCC, 238 mg) were dissolved with 50 ml of anhydrous methylene chloride. The solution was stirred at room temperature overnight and the solvent removed by vaporization. The residue was dissolved in 35 ml of 1,4 dioxane and the insoluble solid was removed by filtration. The solvent was condensed and then precipitated into 100 ml of 50:50/ether:isopropanol. The precipitate was collected by filtration and dried under vacuum. The resulting yield was 3.2 g (78%). ¹H NMR(DMSO-d₆): δ3.5 (br m, PEG), 4.23 (t, —PEGOCH₂CH₂)—CO— (Compound V)), 1.91 (s, (Compound V)-O¹COCH₃), 2.00 (s, (Compound V)-O²COCH₃), 0.84 (t, (Compound V)-CH₃), 4.77 (s, (Compound V)-CH₂COOPEG), 7.03 (t, Compound V aromatic proton), 6.7 (d+d, Compound V aromatic proton).

### EXAMPLE 3

### Synthesis of mPEG20 kDa-ester-Compound V

A compound of Group 5 wherein each Z₂ is a mPEG having a molecular weight of about 20,000 daltons, referred hereinafter as “mPEG-20 k”, attached through a group —CO—(CH₂)₂—O—, was prepared in the following manner.

In a round-bottom flask, Compound V (200 mg) and sodium hydroxide (21 mg) were mixed in 40 ml of anhydrous acetonitrile. 90 mg of benzyl bromide was added to the suspension and the mixture was refluxed for two days. The solid was removed by filtration, the solvent condensed, and the residue dried under vacuum. An oily product, Compound V-benzyl ester, was obtained. The yield was 210 mg (100%). ¹H NMR(DMSO-d₆): δ7.37 (s, C₆H₅—CH₂—OCO— (Compound V)), 5.19 (s, C₆H₅—CH₂—OCO— (Compound V)), 4.83 (s, (Compound V)-CH₂COOBz), 4.49 (d, (Compound V)-OH¹), 4.24 (d, (Compound V)-OH²), 0.864 (t, (Compound V)-CH₃), 7.025 (t, Compound V aromatic proton), 6.7 (d+d, Compound V aromatic proton).

In a round-bottom flask, mPEG-20 k (3 g), Compound V-benzyl ester (prepared in the previous step, 100 mg), HOBT (3 mg), DMAP (25 mg) and DCC (42 mg) were dissolved in 40 ml of anhydrous methylene chloride. The solution was stirred at room temperature overnight, and the solvent was removed by vaporization. The residue was dissolved in 30 ml of 1,4-dioxane and the insoluble solid was removed by filtration. The solvent was condensed, and then precipitated into 100 ml of 50:50/ether:isopropanol. The precipitate, mPEG-Compound V benzyl ester, was collected by filtration and dried under vacuum. The yield was 2.7 g (90%). ¹H NMR(DMSO-d₆): δ3.5 (br m, PEG), 2.48 (t, mPEG-OCH₂CHCOO— (Compound V)), 7.35 (s, C₆H₅—CH₂—OCO— (Compound V)), 5.17 (s, C₆H₅—CH₂—OCO— (Compound V)), 4.83 (s, (Compound V)-CH₂COOBz), 0.857 (t, (Compound V)-CH₃), 7.025 (t, Compound V aromatic proton), 6.7 (d+d, Compound V aromatic proton).

A solution of mPEG-Compound V benzyl ester (obtained in the previous step, 2.7 g) in 1,4-dioxane (30 ml) was hydrogenated with H₂ (2 atm pressure) and 1 gram of Pd/C (10%) overnight. The catalyst was removed by filtration and the catalyst was washed with fresh methylene chloride. The combined solution was condensed by rotary evaporation and the residual syrup was added into 300 ml of ethyl ether. The product was collected by filtration and dried under vacuum. The yield was 2 gram (74%). ¹H NMR(DMSO-d₆): δ3.5 (br m, PEG), 2.48 (t, mPEG-OCH₂CH₂COO— (Compound V)), 4.61 (s, mPEG- (Compound V)-CH₂COOH), 0.857 (t, (Compound V)-CH₃), 7.025 (t, Compound V aromatic proton), 6.7 (d+d, Compound V aromatic proton).

### EXAMPLE 4

### Evaluation of the Antimetastatic Effects of mPEG5 kDa-ester-Compound V

A compound in accordance with the present invention, mPEG5 Kda-ester-Compound V, was prepared through a process similar to the one described in Example 3 except an mPEG group having a molecular weight of about 5,000 daltons (mPEG-5 k) was attached through a group —CO—(CH₂)₂—O. The compound as described herein was tested in an assay to measure the inhibitory effects of the compound on collagen degradation activity.

As described herein, the assay utilizes a thin coating of collagen labeled with a fluorescent dye probe to form a scaffold collagen matrix, and tumor cells. The scaffold collagen matrix simulates the extracellular environment that is attacked by tumor cells during metastasis. The tumor cells attack the matrix by secreting proteolytic enzymes that break the collagen into peptide fragments. The peptide fragments release the fluorescent dye probe, and fluorescent light is emitted. The observed light intensity of the fluorescence correlates proportionally with the collagen degradation activity of the tumor cells.

Procedure

The tumor cells used in the test samples were generated from a human amelanotic melanoma cell line (LOX). A solution of a Type I collagen was prepared at a concentration of about 3.56 mg/ml. The collagen solution was used to form a coating of a monolayer of Type I collagen on a 96-well microtiter plate. The coating of Type I collagen on the microtiter plate was prepared by pipetting and polymerizing each well with a mixture of the Type I collagen solution, DMEM, and sterilized water in a volumetric ratio of 1:2:1. The collagen-coated microtiter plate was incubated at a temperature of about 37° C. for about 1 hour and air dried overnight. A stock fluorescent dye solution (Bodipy FL-C5 SE dye) having a concentration of about 5 mg/ml in dimethyl sulfoxide (DMSO) was obtained from Molecular Probes, Inc. of Eugene, Oreg. The fluorescent dye solution was diluted in a PBS buffer to a concentration of about 1.25 μg/ml. 100 μl of the diluted fluorescent dye solution was added to each well.

The microtiter plate containing the collagen coating and the dye was incubated at room temperature for a time sufficient to allow conjugation, typically for about 1 hour. The microtiter plate was washed five times with phosphate buffer solution (NaCl, 150 mM, Na₂HPO₄, 16 mM, NaH₂PO₄, 4 mM, pH 7.3) to remove excess reagents. The LOX cells (10⁴ cells/well) were added to the microtiter plate.

Monomethyl-terminated PEG 5,000 Da ester-linked Compound V (mPEG5 kDa-ester-Compound V), a compound similar to the one produced in Example 3 except for the particular molecular weight of the PEG component, was added to each well (excluding the control) at varying concentrations ranging from about 2 to 100 μg/mL. The cells were maintained at a temperature of about 37° C. overnight in a carbon dioxide incubator. Fluorescence readings were made using a microtiter plate fluorescence reader set at 485 nm/538 nm (Ex/Em).

Results

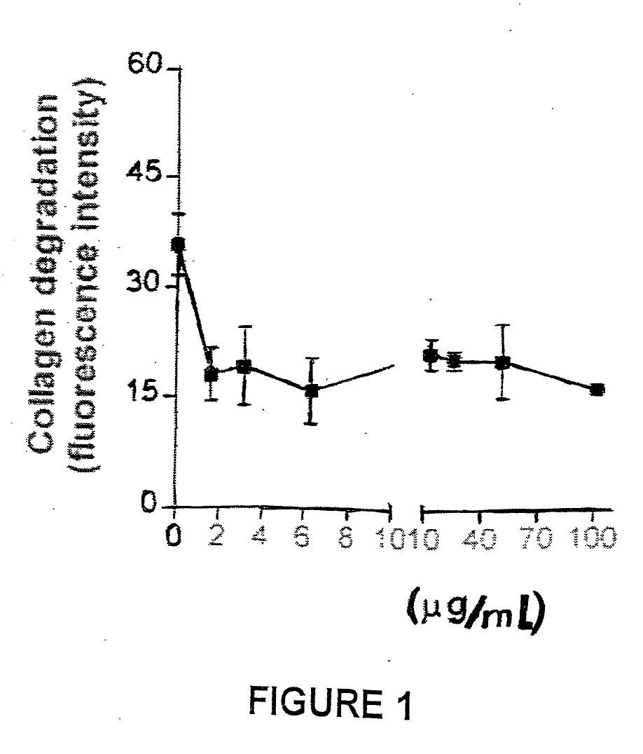

The results of the study are shown in FIG. 1. As indicated above, the level of collagen degradation activity can be determined by measuring the amount of fluorescence intensity observed in each of the test samples. The test samples containing mPEG5 kDa-ester-Compound V demonstrated significant reductions of collagen degradation activity even at the lowest dosages (2 μg/ml) as compared to the test sample containing no mPEG5 kDa-ester-Compound V (control). These results indicate that mPEG5 Kda-ester-Compound V possesses inhibitory effects on collagen degradation activity of LOX cells measurable at dosages of 2 μg/ml and above.

### EXAMPLE 5

### Synthesis of Compound V Diacetate

A compound of Group 5 wherein Z₁ is hydrogen, X is O and each Z₂ is an acetyl group, was prepared in the following manner.

In a round-bottom flask, Compound V (400 mg) and pyridine (200 μl) were mixed in 35 ml of anhydrous methylene chloride. 500 μl of acetic anhydride was added to the suspension. The mixture became homogenous in a few hours and the solution was stirred at room temperature overnight. The solvent was condensed and phosphate buffer (0.1 M, pH 7.4) was added to the residue. The mixture was rapidly stirred for 30 minutes, and the mixture was extracted with methylene chloride three times. The combined organic phase was dried over sodium sulfate, and the solvent was removed by vaporization. An oily product, Compound V Diacetate, was obtained. The yield was 340 mg (80%). ¹H NMR(DMSO-d₆): 1.91 (s, (Compound V)-O¹COCH₃), 2.00 (s, (Compound V)-O²COCH₃), 0.84 (t, (Compound V)-CH₃).

### EXAMPLE 6

### Synthesis of mPEG20 kDa-ester-Compound V Diacetate

A compound of Group 4 wherein Z₁ is a mPEG with a molecular weight of about 20,000 daltons (mPEG-20 k), X is O and each Z₂ is an acetyl group, was prepared in the following manner.

In a round bottom-flask, mPEG-20 k daltons (5.2 g), Compound V diacetate (140 mg), 1-hydroxybenzyltriazole (HOBT, 35 mg), 4-(dimethylamino)pyridine (DMAP, 30 mg) and dicyclo-hexylcarbodiimide (DCC, 75 mg) were dissolved in 60 ml of anhydrous methylene chloride. The solution was stirred at room temperature overnight and the solvent removed by vaporization. The residue was mixed with 35 ml of 1,4 dioxane and the insoluble solid was removed by filtration. The solution was concentrated under vacuum and then added to 200 ml of 50:50/ether:isopropanol. The resulting precipitate was collected by filtration and dried under vacuum. Yield: 4.8 g (92%). ¹H NMR (DMSO-d₆): δ3.5 (br m, PEG), 4.23 (t, —PEGOCH₂CH₂O—CO— (Compound V)), 1.91 (s, (Compound V)-OCOCH₃), 2.00 (s, (Compound V)-OCOCH3), 0.84 (t, (Compound V)-CH3), 4.77 (s, (Compound V)-CH₂COOPEG), 7.03 (t, Compound V aromatic proton), 6.7 (d+d, Compound V aromatic proton).

### EXAMPLE 7

### Synthesis of mPEG350 Da-amide-Compound V Diacetate

A compound of Group 4 wherein Z₁ is a mPEG with a molecular weight of about 350 daltons (mPEG-350), X is NH and each Z₂ is an acetyl group, was prepared in the following manner.

In a round-bottom flask, Compound V (400 mg), mPEG-350 amine (360 mg), HOBT (15 mg), and DCC (267 mg) were mixed with 20 ml of anhydrous methylene chloride and the mixture was stirred at room temperature overnight. The insoluble solid was removed by filtration and the organic solution was washed with 5 wt % sodium bicarbonate solution. The organic phase was dried over sodium sulfate and the solvent removed under vacuum. The resulting product was dissolved in 10 ml of acetonitrile and the insoluble solid was removed by filtration. To the solution was added acetic anhydride (3 ml) and pyridine (0.3 ml). The resulting solution was heated at 40° C. overnight. To the solution was added 300 ml of 5 wt % sodium bicarbonate solution and the mixture was stirred 30 minutes at room temperature. The mixture was extracted with methylene chloride and the organic phase was washed with phosphate buffer (0.1 M, pH 2) and dried over sodium sulfate. The solvent was removed and the product dried under vacuum. The yield was 600 mg (70%). ¹H NMR (DMSO-d₆): δ3.5 (br m, PEG), 7.897 (t, —PEGNH—CO-(Compound V)), 1.91 (s, (Compound V)-O¹COCH₃), 2.00 (s, (Compound V)-O²COCH₃), 0.864 (t, (Compound V)-CH₃), 4.436 (s, (Compound V)-CH₂CONHPEG), 7.045 (t, Compound V aromatic proton), 6.7 (d+d, Compound V aromatic compound).

### EXAMPLE 8

### Synthesis of mPEG 350 Da-ester-Compound V Diacetate

A compound of Group 4 wherein Z₁ is a mPEG with a molecular weight of about 350 daltons (mPEG-350), X is O and each Z₂ is an acetyl group, was prepared in the following manner.

In a round-bottom flask, Compound V (3 g) and triethylamine (TEA, 1.5 μl) were mixed in 100 ml of anhydrous acetonitrile. To the solution was added 3 ml of acetyl chloride. The mixture was stirred at room temperature overnight. The solution was then mixed with 5 wt % sodium bicarbonate solution and stirred 30 minutes at room temperature. The aqueous phase was extracted with methylene chloride. The organic phase was washed with phosphate buffer (0.1 M, pH 2) and then dried over sodium sulfate. The yield was 3.3 g (80%). ¹H NMR(DMSO-d₆): 1.91 (s, (Compound V)-O¹COCH₃), 2.00 (s, (Compound V)-O²COCH₃), 0.84 (t, (Compound V)-CH₃).

In a round-bottom flask, mPEG-350 (550 mg), Compound V diacetate from the previous step (750 mg), HOBT (60 mg), DMAP (150 mg), and DCC (375 mg) were dissolved in 30 ml of anhydrous methylene chloride. The solution was stirred at room temperature overnight. The insoluble solid was removed by filtration and the solution was washed with 5 wt % sodium bicarbonate solution and phosphate buffer (0.1 M, pH 2). The organic phase was dried over sodium sulfate and concentrated under vacuum. The resulting product was dissolved in 10 ml of acetonitrile and the insoluble solid was removed by filtration. The solvent was removed by vaporization and the product was obtained in the form of clear oil. The yield was 1 g (76 %). ¹H NMR(DMSO-d₆): δ3.5 (br m, PEG), 4.23 (t, —PEGOCH₂CH₂O—CO-(Compound V)), 1.91 (s, (Compound V)-O¹COCH₃), 2.00 (s, (Compound V)-O²COCH₃), 0.84 (t, (Compound V)-CH₃), 4.77 (s, (Compound V)-CH₂COOPEG), 7.03 (t, Compound V aromatic proton), 6.7 (d+d, Compound V aromatic proton).

### EXAMPLE 9

### Comparison of Collagen Degradation Inhibiting Effects of Pegylated Compound,

### Compound V and a Hydroxamic Acid-based Inhibitor of Matrix Metalloproteinase on Human Amelanotic Melanoma Cells

A study using test samples prepared from a Type I collagen matrix having tumor cells generated from a human amelanotic melanoma cell line (LOX) seeded thereon, was implemented to evaluate and compare the pharmacokinetic effects of Compound V, mPEG5 kDa-ester-Compound V or pegylated Compound V, and BATIMASTAT®, a hydoxamic acid metalloproteinase inhibitor, or “MMP inhibitor” hereinafter which is known to be useful for inhibiting metastasis. See, Zucker et al, Critical appraisal of the use of matrix metalloproteinase inhibitors in cancer treatment, Oncogene (2000) 19, 6642-6650. The MMP inhibitor was obtained from British Biotech of Oxford, UK. The test compounds were assayed and analyzed to measure their collagen degradation inhibiting effects on the human amelanotic melanoma cells.

Collagen Degradation Inhibition Assay

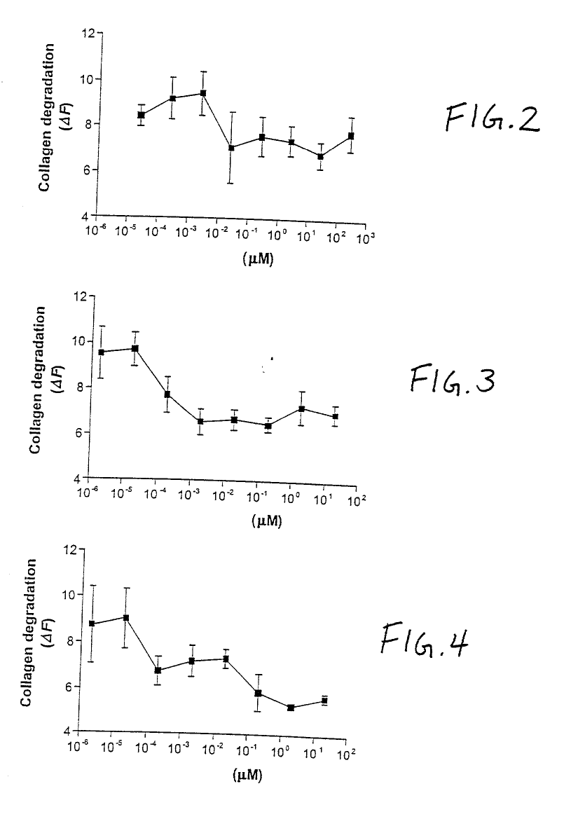

The collagen degradation inhibition assay was performed using the same procedures and steps described in Example 4. The LOX cells were cultured and incubated in a carbon dioxide incubator at 37° C. for 16 hours. The cells were seeded on the thin film of collagen matrix supported on a microtiter plate. The test compounds were added to the corresponding tissue cultures at dosages varying from about 10⁻⁶ to 10³ μM. The collagen degradation activity of the LOX cells was detected through the cleavage and release of fluorescent collagen peptides from the labeled collagen matrix film as described above. A positive fluorescence signal, ΔF indicates the presence of collagen degradation activity. The collagen degradation activity assay was performed four times (n=4) and its results are represented as mean±SEM values in FIGS. 2, 3, and 4.

Referring to FIG. 2, the graph shows the results of the collagen degradation activity assay for Compound V. The assay showed that compound V inhibited collagen degradation activity by LOX cells at a dose amount of about 2.5×10⁻³ μM (IC₅₀=1×10⁻² μM). Referring to FIG. 3, the graph shows the results of the collagen degradation activity assay for pegylated compound V. The assay showed that pegylated compound V inhibited collagen degradation activity by LOX cells at dosages of over 2×10⁻⁵ μM (IC₅₀=2×10⁻⁴ μM). Referring to FIG. 4, the graph shows the results of the collagen degradation activity assay for the MMP inhibitor. The assay showed that the MMP inhibitor inhibited collagen degradation activity by LOX cells at dosages of over 10⁻⁴ μM (IC₅₀=2×10⁻⁴ μM).

Conclusion

The results of the collagen degradation inhibition assay indicate that the pegylated compound V exhibited substantially similar collagen degradation inhibiting effects over the range of dosages tested as observed in compound V and the known MMP inhibitor.

### EXAMPLE 10

### Comparison of Cell Toxicity of Pegylated Compound V, Compound V and a Hydroxamic Acid-based Inhibitor of Matrix Metalloproteinase on Human Amelanotic Melanoma Cells

A study using test samples prepared from a Type I collagen matrix having tumor cells generated from a human amelanotic melanoma cell line (LOX) seeded thereon, was implemented to evaluate and compare the pharmacokinetic effects of Compound V, mPEG5 kDa-ester-Compound V or pegylated Compound V, and BATIMASTAT®, a hydoxamic acid metalloproteinase inhibitor, or “MMP inhibitor” hereinafter. The MMP inhibitor was obtained from British Biotech of Oxford, UK. The test compounds were assayed and analyzed to measure their cell toxicity on human amelanotic melanoma cells.

Cell Toxicity Assay

The test compounds were assayed to evaluate and compare their cell toxicity on the LOX cells. A live cell toxicity assay marketed as LIVE/DEAD Viability/Cytotoxicity Kit by Molecular Probes, Inc. of Eugene, Oreg., was obtained for the test. The assays were carried out in accordance with the manufacturers' instructions using the LOX cells. The LOX cells (10⁴/well) were cultured and incubated in a carbon dioxide incubator at 37° C. overnight and then gently washed with PBS.

Calcein Am (2 μM), a fluorescent dye available from Molecular Probes, Inc. for staining live cells, was added to the LOX cells. The Calcein-stained LOX cells were incubated at room temperature for about an hour. The test compounds were added to the LOX cell cultures at dosages varying from about 10⁻⁶ to 10³ μM. The LOX cell cultures were incubated overnight. A positive fluorescence signal from the assay indicates the presence of viable cells in the test sample with the strength of the signal being proportional to the number of viable cells present.

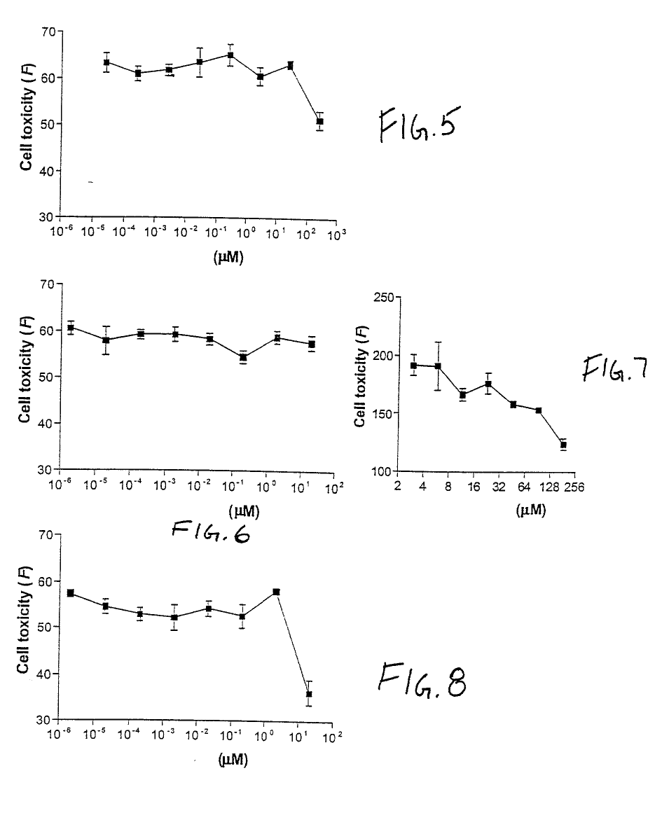

The live cell toxicity assay was carried out four times (n=4) for each test compound. The results of the assay for each compound are represented as mean±SEM values in FIGS. 5, 6, 7, and 8. With reference to FIG. 5, the graph shows that compound V exhibited cell toxicity and inhibited cell viability at dosages of over 10 μM. Referring to FIGS. 6 and 7, each of the corresponding graphs shows that pegylated compound V exhibited little or no cell toxicity below 16 μM. Referring to FIG. 8, the graph shows that the MMP inhibitor exhibited marked cell toxicity at dosages of over 10 μM.

Conclusion

The results of the cell toxicity assay indicate that the pegylated compound V exhibited similar cell toxicity to compound V over the range of dosages. However, the pegylated compound V exhibited slightly lower cell toxicities compared to the MMP inhibitor at dosages below 1 μM, and significantly lower cell toxicities at dosages greater than 1 μM.

### EXAMPLE 11

### Comparison of Apoptosis-Inducing Effects of Pegylated Compound V, Compound V and a Hydroxamic Acid-based Inhibitor of Matrix Metalloproteinase on Human Amelanotic Melanoma Cells

A study using test samples prepared from a Type I collagen matrix having tumor cells generated from a human amelanotic melanoma cell line (LOX) seeded thereon, was implemented to evaluate and compare the pharmacokinetic effects of Compound V, mPEG5 kDa-ester-Compound V or pegylated Compound V, and BATIMASTAT®, a hydoxamic acid metalloproteinase inhibitor, or “MMP inhibitor” hereinafter. The MMP inhibitor was obtained from British Biotech of Oxford, UK. The test compounds were assayed and analyzed to measure their apoptosis-inducing effects on human amelanotic melanoma cells.

Apoptosis Assay

The test compounds were assayed to evaluate and compare their apoptosis-inducing effects on the LOX cells. An apoptosis assay marketed as Vybrant Apoptosis Assay Kit #4 from Molecular Probes, Inc., was obtained for the test. The assays were carried out in accordance with the manufacturers' instructions using the LOX cells. The LOX cells (10⁴/well) were cultured and incubated in a carbon dioxide incubator at 37° C. overnight and then gently washed with PBS.

YO-PRO (2 μM), a fluorescent dye available from Molecular Probes, Inc., for staining apoptotic cells, was added to the LOX cells. The YO-PRO-stained LOX cells were incubated at room temperature for about 20 minutes. The test compounds were added to the second group of LOX cell cultures at dosages varying from about 10⁻⁶ to 10³ μM. The LOX cell cultures were incubated overnight. A positive fluorescence signal indicates the presence of apoptotic cells in the test sample with the strength of the signal being proportional to the number of apoptotic cells present.

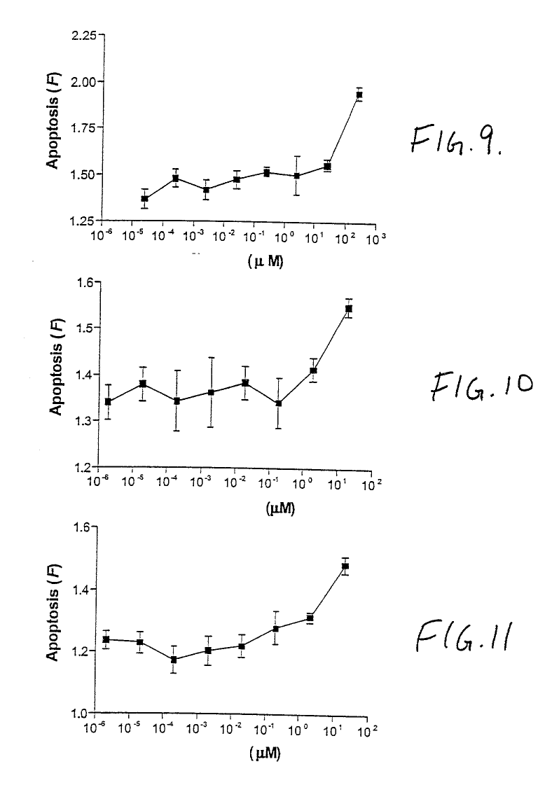

The apoptosis assay was performed four times (n=4) for each test compound. The results of the assay for each compound are represented as mean±SEM values in FIGS. 9, 10, and 11. With reference to FIG. 9, the graph shows that compound V caused LOX cells to enter apoptosis at dosages over 10 μM. The graph further shows a marked increase in the incidence of apoptosis induced by compound V at dosages of over 100 μM. Referring to FIG. 10, the graph shows that pegylated compound V caused the LOX cells to enter apoptosis at dosages of about 2 μM and above. The graph further shows a marked increase in the incidence of apoptosis induced by pegylated compound V at dosages of over 10 μM. Referring to FIG. 11 the graph shows that the MMP inhibitor caused the LOX cells to enter apoptosis at dosages of over 2 μM. The graph further shows a marked increase in the incidence of the MMP inhibitor-induced apoptosis at dosages of over 10 μM.

Conclusion

The results of the apoptosis assay indicate that the pegylated compound V exhibited substantially similar apoptosis-inducing effects on human amelanotic melanoma cells over the range of dosages tested as observed in compound V and the known MMP inhibitor.

What is claimed is:

1. A pharmaceutical composition comprising a cancer-treating effective amount of at least one compound of Formulas Ia or Ib
[P-T]ₙ-Z Ia
P-[T-Z]ₙ Ib
wherein
P is a prostaglandin compound or analog thereof, T is an active group of P, and Z is a pharmaceutically acceptable group which is bound to T and which slows the rate at which the prostaglandin compound is metabolized;
n is an integer of at least 1, and pharmaceutically acceptable salts or esters thereof; and
a pharmaceutically acceptable carrier.

2. The pharmaceutical composition of claim 1 wherein T is selected from the group consisting a carboxyl group, a hydroxyl group, a carbonyl group, an oxidized carbohydrate, and a mercapto group.

3. The pharmaceutical composition of claim 1 wherein T is a carboxyl group or a hydroxyl group.

4. The pharmaceutical composition of claim 1 wherein Z is a pharmaceutically acceptable polymer or an acetyl group.

5. The pharmaceutical composition of claim 4 wherein the pharmaceutically acceptable polymer is selected from the group consisting of polyalkylene oxides, dextran, polyvinyl pyrrolidones, polyacrylamides, polyvinyl alcohols, and carbohydrate based polymers.

6. The pharmaceutical composition of claim 5 wherein the polyalkylene oxides are selected from polyethylene glycols.

7. The pharmaceutical composition of claim 6 wherein the molecular weight of the polyethylene glycols is from about 200 to 80,000.

8. The pharmaceutical composition of claim 7 wherein the molecular weight of polyethylene glycols is from about 2,000 to 42,000.

9. The compounds of claim 8 wherein the molecular weight of the polyethylene glycols is from about 5,000 to 25,000.

10. The pharmaceutical composition of claim 1 wherein said compound has the structure of Formula II 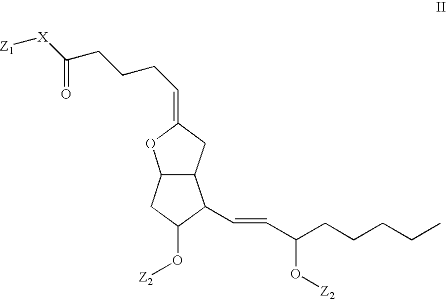
wherein
Z₁ and Z₂ are each independently selected from the group consisting of hydrogen, a pharmaceutically acceptable polymer and an acetyl group with the proviso that at least one of Z₁ and Z₂ are not hydrogen, and X is selected from O and NH, and pharmaceutically acceptable salts or esters thereof.

11. The pharmaceutical composition of claim 10 wherein Z₁ is a pharmaceutically acceptable polymer, X is selected from O and NH and each Z₂ is independently selected from hydrogen and an acetyl group.

12. The pharmaceutical composition of claim 10 wherein Z₁ is hydrogen, X is selected from O and at least one Z₂ is a pharmaceutically acceptable polymer attached to the oxygen atom through an ester group.

13. The pharmaceutical composition of claim 10 wherein Z₁ is a pharmaceutically acceptable polymer, X is selected from O and NH and at least one Z₂ is a pharmaceutically acceptable polymer attached to the oxygen atom through an ester group.

14. The pharmaceutical composition of claim 1 wherein said compound has the structure of Formula III 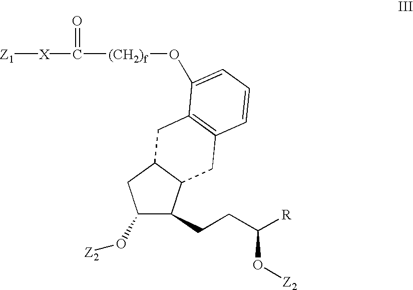
wherein
Z₁ and Z₂ are each independently selected from the group consisting of hydrogen, a pharmaceutically acceptable polymer and an acetyl group with the proviso that at least one of Z₁ and Z₂ are not hydrogen,
f is an integer of from 1 to 3;
X is selected from O and NH; and
R is selected from hydrogen and an alkyl group, and pharmaceutically acceptable salts or esters thereof.

15. The pharmaceutical composition of claim 14 wherein R is an alkyl group having 1-6 carbon atoms.

16. The pharmaceutical composition of claim 14 wherein Z₁ is a pharmaceutically acceptable polymer, X is selected from O and NH and each Z₂ is independently selected from hydrogen and an acetyl group.

17. The pharmaceutical composition of claim 14 wherein Z₁ is hydrogen, X is O and each Z₂ is an acetyl group or a pharmaceutically acceptable polymer attached to the oxygen atom through an ester or an ether group.

18. The pharmaceutical composition of claim 14 wherein Z₁ is a pharmaceutically acceptable polymer, X is selected from O and NH and each Z₂ is a pharmaceutically acceptable polymer attached to the oxygen atom through an ester or an ether group.

19. The pharmaceutical composition of claim 10 wherein the pharmaceutically acceptable polymer is a polyethylene glycol having a molecular weight of from about 200 to 80,000.

20. The pharmaceutical composition of claim 19 wherein the molecular weight of the polyethylene glycol is from about 2,000 to 42,000.

21. The pharmaceutical composition of claim 14 wherein the pharmaceutically acceptable polymer is a polyethylene glycol having a molecular weight of from about 200 to 80,000.

22. The pharmaceutical composition of claim 21 wherein the molecular weight of the polyethylene glycol is from about 2,000 to 42,000.

23. The pharmaceutical composition of claim 14 wherein Z₁ is a methyl terminated polyethylene glycol having a molecular weight of about 5,000, X is NH and each Z₂ is hydrogen.

24. The pharmaceutical composition of claim 14 wherein Z₁ is a methyl terminated polyethylene glycol having a molecular weight of about 5,000, X is O and each Z₂ is an acetyl group.

25. The pharmaceutical composition of claim 14 wherein Z₁ is hydrogen and each Z₂ is a methyl terminated polyethylene glycol having a molecular weight of about 20,000 attached to the oxygen atom through a group —O—(CH₂)₂—CO—.

26. The pharmaceutical composition of claim 14 wherein Z₁ is hydrogen and each Z₂ is an acetyl group.

27. The pharmaceutical composition of claim 14 wherein Z₁ is a methyl terminated polyethylene glycol having a molecular weight of about 20,000, X is O, and each Z₂ is an acetyl group.

28. The pharmaceutical composition of claim 14 wherein Z₁ is a methyl terminated polyethylene glycol having a molecular weight of about 20,000, X is NH and each Z₂ is hydrogen.

29. The pharmaceutical composition of claim 14 wherein Z₁ is a methyl terminated polyethylene glycol having a molecular weight of about 20,000, X is NH and each Z₂ is an acetyl group.

30. The pharmaceutical composition of claim 14 wherein Z₁ is a methyl terminated polyethylene glycol having a molecular weight of about 5,000, X is NH and each Z₂ is an acetyl group.

31. The pharmaceutical composition of claim 14 wherein Z₁ is a methyl terminated polyethylene glycol having a molecular weight of about 350, X is NH and each Z₂ is an acetyl group.

32. The pharmaceutical composition of claim 14 wherein Z₁ is a methyl terminated polyethylene glycol having a molecular weight of about 350, X is O and each Z₂ is an acetyl group.

33. The pharmaceutical composition of claim 14 wherein said compound has the structure of Formula IV 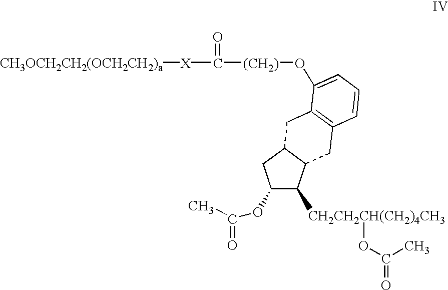
wherein
a is from about 6 to 600.

34. The pharmaceutical composition of claim 1, wherein P is a PGE-type prostaglandin.

35. A method of treating cancer comprising administering to a warm-blooded animal including humans afflicted with cancer, a cancer-treating effective amount of the pharmaceutical composition of claim 1.

36. The method of claim 35 comprising administering said pharmaceutical composition in a dosage amount of from about 0.1 to 500 mg/kg/day to said warm-blooded animal.

37. The method of claim 35 comprising administering said pharmaceutical composition intravenously to said warm-blooded animal.

38. The method of claim 35 comprising administering said pharmaceutical composition subcutaneously to said warm-blooded animal.

39. The method of claim 35 comprising administering said pharmaceutical composition by inhalation to said warm-blooded animal.

40. The method of claim 35 comprising administering said pharmaceutical composition orally to said warm-blooded animal.

41. A method of inhibiting metastasis in a warm-blooded animal including humans afflicted with cancer, said method comprising administering to the warm-blooded animal a metastasis-inhibiting effective amount of the pharmaceutical composition of claim 1.

42. The method of claim 41 comprising administering said pharmaceutical composition in a dosage amount of from about 0. 1 to 500 mg/kg/day to said warm-blooded animal.

43. The method of claim 41 comprising administering said pharmaceutical composition intravenously to said warm-blooded animal.

44. The method of claim 41 comprising administering said pharmaceutical composition subcutaneously to said warm-blooded animal.

45. The method of claim 41 comprising administering said pharmaceutical composition by inhalation to said warm-blooded animal.

46. The method of claim 41 comprising administering said pharmaceutical composition orally to said warm-blooded animal.

47. A method of inhibiting collagen degradation induced by metastasizing cancer cells in a warm-blooded animal including humans afflicted with cancer, said method comprising administering to the warm-blooded animal a collagen degradation inhibiting effective amount of the pharmaceutical composition of claim 1.

48. The method of claim 47 comprising administering said pharmaceutical composition in a dosage amount of from about 0.1 to 500 mg/kg/day to said warm-blooded animal.

49. The method of claim 47 comprising administering said pharmaceutical composition intravenously to said warm-blooded animal.

50. The method of claim 47 comprising administering said pharmaceutical composition subcutaneously to said warm-blooded animal.

51. The method of claim 47 comprising administering said pharmaceutical composition by inhalation to said warm-blooded animal.

52. The method of claim 47 comprising administering said pharmaceutical composition orally to said warm-blooded animal.
